(19) United States
(12) Patent Application Publication
(10) Pub. No.: US 2006/0167435 A1
(43) Pub. Date: Jul. 27, 2006

(54) Transscleral drug delivery device and related methods

(76) Inventors: Anthony P. Adamis, Bronxville, NY (US); Joan W. Miller, Winchester, MA (US); Mark J. Mescher, West Newton, MA (US); Evangelos S. Gragoudas, Lexington, MA (US); Jeffrey T. Borenstein, Holliston, MA (US)

Correspondence Address: GOODWIN PROCTER LLP;PATENT ADMINISTRATOR, EXCHANGE PLACE, BOSTON, MA, 02109-2881, US

(21) Appl. No.: 10/545,726

(22) PCT Filed: Feb. 17, 2004
(86) PCT No.: PCT/US04/04625

§ 371 Date: Mar. 23, 2006

Related U.S. Application Data
(60) Provisional application No. 60/447,971, filed on Feb. 18, 2003.

(51) Int. Cl. A61M 31/00

(52) U.S. Cl. 604/500

(57) ABSTRACT

The invention provides a low-profile, dome-shaped body for attachment to a scleral surface of an eye and defining an internal cavity for receiving a drug or other pharmaceutically active agent. The device has an opening for controllably delivering the drug into the eye at therapeutically effective concentrations over a prolonged period of time. When attached, the device does not affect or otherwise restrict movement of the eye. Features of the invention include an optional drug inlet port and puncture guard, both designed for refilling the device while preventing a needle inserted through the inlet port from contacting the sclera.

### CROSS-REFERENCE TO RELATED APPLICATION

This application claims the benefit of and priority to U.S. Provisional Application Ser. No. 60/447,971, filed Feb. 18, 2003, the disclosure of which is incorporated by reference herein.

### FIELD OF THE INVENTION

This invention relates generally to transscleral drug delivery and, mote particularly, to an implantable device for transsclerally delivering a drug to the vitreal cavity of a mammalian eye, and to a method for introducing a drug into the vitreal cavity using the device.

### BACKGROUND OF THE INVENTION

The way a particular drug is administered to a recipient can significantly affect the efficacy of the drug. For example, some therapies, in order to be optimal, require that the drug be administered locally to a particular target site. Furthermore, some of those drugs need to be present at the target site for a prolonged period of time to exert maximal effect

One approach for achieving localized drug delivery involves injection of drug directly into the site of desired drug activity. Unfortunately, this approach may require periodic injections of drug to maintain an effective drug concentration at the target site. In order to prolong the existence at the target site, the drug may be formulated into a slow release formulation (see, for example, Langer (1998) Nature 392, Supp. 5-10). For example, the drug can be conjugated with polymers which, when administered to an individual, are then degraded, for example, by proteolytic enzymes or by hydrolysis, to gradually release drug into the target site. Similarly, drug can be trapped throughout insoluble matrices. Following administration, drug then is released via diffusion out of, or via erosion of, the matrices. Alternatively, drug can be encapsulated within a semi-permeable membrane or liposome. Following administration, the drug is released either by diffusion through the membrane or via breakdown of the membrane. However, problems associated with localized drug injection can include, for example, repeated visits to a health care professional for repeated injections, difficulty in stabilizing drugs within slow release formulations, and the control of the concentration profile of the drug over time at the target site.

Another approach for localized drug delivery includes the insertion of a catheter to direct the drug to the desired target location. The drug can be pushed along the catheter from a drug reservoir to the target site via, for example, a pump or gravity feed. Typically, this approach employs an extracorporeal pump, an extracorporeal drug reservoir, or both an extracorporeal pump and extracorporeal drug reservoir. Disadvantages can include, for example, the risk of infection at the catheter's point of entry into the recipient's body, and that because of their size the pump and/or the reservoir may compromise the mobility and life style of the recipient.

Over the years, implantable drug delivery devices have been developed to address some of the disadvantages associated with localized injection of drug or the catheter-based procedures. A variety of implantable drug delivery devices have been developed to date.

One type of implantable drug delivery device includes the osmotically driven device. A variety of osmotic drug delivery devices are known in the art For example, one such device is available commercially from Durect Corp. (Cupertino, Calif.) under the tradename DUROS®. Similarly another device is available from ALZA Scientific Products (Mountain View, Calif.), under the tradename ALZET®. In some devices, the influx of fluid into the device causes an osmotically active agent to swell. The swelling action can then be employed to push drug initially stored in a reservoir out of the device. DUROS® pumps reportedly deliver up to 200 mg of drug at rates as low as 0.5 μL per day. However, osmotic pumps stop working when the osmotic engine in the device or drug reservoir becomes exhausted.

In addition to osmotically driven drug delivery devices, a variety of mechanical and electrochemical devices have been developed to date. U.S. Pat. No. 3,692,027, for example, describes an implantable, electro-mechanical drug delivery device. The device includes, within a fluid-impermeable and sealed casing, a watch-type drive mechanism that drives a circular wheel The wheel contains a plurality of cavities, all of which apparently are radially disposed in a single plane about the circumference of the wheel Once the drug-containing cavity moves into alignment with an aperture through the casing, a piston associated with the cavity ejects medicine out of the cavity and through the aperture. This type of device can be quite large in size and, therefore, may be unsuitable for implantation into small cavities within the body.

One area where implantable devices capable of delivering a drug to the target site for a prolonged period of time are particularly useful is the field of ophthalmology. Within the past several decades, great advances have been made in the diagnosis and treatment of various ocular disorders. Advances in laser technology and vitreoretinal surgical techniques have significantly improved the prognosis of numerous ocular disorders including, for example, diabetic retinopathy, macular degeneration, and retinal detachment. As the pathology of these and many other ocular disorders is also becoming more clearly understood, significant efforts have been made to identify drugs that, once administered to the eye, can ameliorate one or more symptoms of these disorders. In addition to the numerous antibiotic, antiviral, and antifungal agents currently being used to treat infections of the retina and vitreous, many anti-angiogenic drugs, anti-inflammatory drugs and anticancer drugs, for example, topical and periocular steroids, have been shown to be useful in treating ocular disorders. As another example, an anti-sense based therapeutic known as Vitravene™ has been approved in the U.S. for the treatment of cytomegalovirus retinitis (see, for example, de Smet et al. (1999) OCULAR IMUNOL. INFL. 7: 189-198). In addition, an anti-vascular endothelial growth factor (VEGF) antibody and an anti-VEGF aptamer currently are being tested as agents for the treatment of the neovascular form of age-related macular degeneration (see, for example, Guyer et al. (2002) RETINA 22:143-152).

Unfortunately, the delivery of drugs into the interior of an eye can be problematic. Although some drugs can be administered systemically, for example, orally or intravenously, some of the blood vessels in the retina (and other parts of the central nervous system) are relatively impermeable to many drugs. Accordingly, very high concentrations of drug may be required in the systemic circulation to generate therapeutically effective dosages in the eye. This may create significant systemic side effects on other organs of the body.

The problems associated with systemic administration may be mitigated by localized administration, for example, via topical application and intravitreal injection. However, both approaches have their own problems. For example, drugs applied topically to the eye, for example, in the form of eye drops, may not penetrate through the cornea well enough to provide therapeutically effective concentrations in the eye. Alternatively, when drugs are injected directly into the vitreous cavity, this procedure itself entails certain risks, such as infection, bleeding, cataract formation, and retinal detachment Furthermore, the majority of the injected drug is often cleared from the vitreous cavity within several days, necessitating multiple injections for prolonged treatment.

Accordingly, a variety of devices have been developed for introducing drugs into the vitreal cavity. U.S. patent application publication no. 2002/0026176, for example, discloses a drug-containing plug that can be inserted through the sclera so that it projects into the vitreous cavity to deliver drug into the vitreous cavity. U.S. Pat. No. 5,443,505 discloses an implantable device for introduction into a suprachoroidal space or an avascular region for sustained release of drug into the interior of the eye. U.S. Pat. Nos. 5,773,019 and 6,001,386 disclose an implantable drug delivery device attachable to the scleral surface of an eye. The device comprises an inner core containing an effective amount of a low solubility agent covered by a non-bioerodible polymer that is permeable to the low solubility agent. During operation, the low solubility agent permeates the bioerodible polymer cover for sustained release out of the device. U.S. Pat. No. 6,416,777 discloses a device comprising a pharmaceutically active agent and having a geometry that facilitates the implantation of the device onto an outer surface of the sclera beneath the inferior oblique muscle such that, during operation, the agent is disposed above the macula. Also known is a drug delivery device that is made of a biodegradable polymer containing dexamethasone steroid that can be inserted into the anterior or posterior chamber via a 20-gauge incision. Another known drug delivery device is a reservoir filled with fluocinolone acetonide that is implanted to the vitreal cavity through a 3.5-mm incision.

Although a variety of implantable drug delivery devices have been developed to date, there is still an ongoing need in the art for reliable, miniturized, implantable drug delivery devices that permit the localized delivery of a drug over a prolonged period of time thereby maintaining the drug at the target site in therapeutic concentrations.

### SUMMARY OF THE INVENTION

Accordingly, it is an object of the present invention to provide a transscleral drug delivery device that overcomes the limitations of known devices and methods. Specifically, it is an object of the present invention to provide for improved delivery of drugs and other pharmacological agents to the vitreous cavity of the eye, especially for treating ocular disorders. Another object of the present invention is to provide a device that permits a drug to be delivered to the vitreous cavity with a single initial surgery and without the need for repeated invasive surgeries or procedures. Yet another object of this invention is to allow replenishment of the drug within an implant already attached to the sclera by injection of the drug into the implant, without surgery or other invasive procedure.

Accordingly, the invention features a low-profile, dome-shaped body for attachment to an exterior scleral surface of a mammalian, for example, a human, eye. The dome-shaped body defines an internal cavity for receiving a drug. The device has an opening for controllably delivering the agent into the eye in therapeutic concentrations over a prolonged period of time. When attached, the device does not substantially affect or otherwise restrict movement of the eye.

In one aspect, the invention provides a transscleral drug delivery device for delivering a drug into a mammalian eye. The device includes a dome member that has a base region and defines a cavity for receiving the drug, and a base plate attached to the base region. The base plate has a sclera-contacting surface generally concave in shape for attaching the device to the scleral surface of the eye. The base plate defines at least one drug outlet port to provide fluid flow communication between the cavity and the scleral surface of the eye when the device is attached to the eye. The drug outlet port is at least 25%, preferably 25% to 50%, of the footprint of the base region. The base plate may optionally be integral with the dome member. In various embodiments, at least one of the dome member and the base plate is fabricated from a biocompatible, non-biodegradable material, for example, a metal. In various embodiments, the device further includes a drug disposed within the cavity of the dome member. The drug outlet port is dimensioned to permit controlled delivery of drug to the outer surface of the eye, which can then diffuse through the sclera to permit a therapeutically effective amount of the drug to accumulate within the interior of the eye.

In one embodiment, the base plate has a first diameter and defines at least one drug outlet port having a second diameter. The second diameter equals at least one half of the first diameter.

The dome member may further define a drug inlet port for introducing the drug into the cavity. In various embodiments, at least a portion of the dome member or the base plate is substantially impenetrable to a needle inserted through the drug inlet port. The device may also include a puncture guard for preventing a needle inserted through the drug inlet port from contacting the scleral surface of the eye. The puncture guard may be disposed adjacent to at least one surface of the base plate, or at least one surface of the dome member, and may be fabricated from a rigid material, for example, a metal.

In another aspect, the invention provides a transscleral drug delivery device for delivering a drug into a mammalian eye. The device includes a dome member that has a base region and defines a cavity for receiving the drug and at least one drug inlet port for introducing the drug into the cavity. The device also includes a base plate attached to the base region. The base plate has a sclera-contacting surface for attaching the device to a scleral surface of the eye and defines a drug outlet port to provide fluid communication between the cavity and the scleral surface of the eye when the device is attached to the scleral surface. The device further includes a puncture guard for preventing a needle inserted through the drug inlet port from contacting the scleral surface. The puncture guard may be disposed adjacent to at least one surface of the base plate, or at least one surface of the dome member, and may be fabricated from a rigid material, for example, a metal. In various embodiments, the device further includes a drug disposed within the cavity of the dome member.

The base plate may optionally be integral with the dome member. In various embodiments, at least one of the dome member and the base plate is fabricated from a biocompatible, non-biodegradable material, for example, a metal. In some embodiments, the material of at least one of the dome member and the base plate is biodegradable.

In yet another aspect, the invention provides a transscleral drug delivery device for delivering a drug into a mammalian eye. The device includes a dome member that has a base region and defines a cavity for receiving the drug, and at least one drug inlet port for introducing the drug into the cavity. The drug inlet port is configured to prevent a needle inserted therethrough from contacting a scleral surface of the eye when the device is attached to the eye. The device also includes a base plate attached to the base region. The base plate has a sclera-contacting surface for attaching the device to a scleral surface of the eye and defines a drug outlet port to provide fluid communication between the cavity and the scleral surface when the device is attached to the eye.

In some embodiments of this aspect of the invention, the drug inlet port is an aperture, which is defined by the dome member, and which has an axis that is orthogonal to the aperture and does not intersect the base plate. In a particular embodiment, the axis is substantially parallel to the base plate. To the extent that the axis orthogonal to the aperture intersects the base plate, the device preferably comprises a substantially rigid base plate and/or a puncture guard to prevent a needle inserted through the drug inlet port from contacting the scleral surface of the eye.

In other embodiments of this aspect of the invention, the drug inlet port includes a generally tubular member that is disposed in an aperture defined by the dome member and defines a lumen having a central longitudinal axis. The central longitudinal axis of the lumen does not intersect the base plate, for example, is substantially parallel to the base plate.

In still others aspects, the invention provides a method of delivering a drug into a mammalian eye. The method includes attaching the transscleral drug delivery device described above to a scleral surface of the eye; and permitting drug disposed within the dome member to exit the cavity and contact the scleral surface. In various embodiments, the methods further include introducing drug into the cavity.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A depicts a top view of a transscleral drug delivery device according to one embodiment of the invention attached to a scleral surface of a human eyeball;

FIG. 1B depicts a cross-section of the embodiment shown in FIG. 1A taken along line A-A;

FIG. 2 depicts a dome member of the transscleral drug delivery device having a base region according to one embodiment of the invention;

FIGS. 3A-3C depict a dome member of the transscleral drug delivery device having an drug inlet port according to the embodiments of the invention; and

FIGS. 4A-4C depict a dome member of the transscleral drug delivery device having a puncture guard according to the embodiments of the invention.

### DETAILED DESCRIPTION

It has been discovered that certain drugs, when applied to the outer surface of an eye, can traverse the sclera and enter the interior of the eye (see, PCT/US00/00207 and Ambati et al. (2000) INVEST. OPHTHAL. VIS. SCI. 41:1181-1185). More specifically, it has been found that large molecules, for example, immunoglobulin G can diffuse across the sclera of rabbit eyes in a manner consistent with porous diffusion through a fiber matrix (Ambati et at (2000) supra). This observation has led to the possibility of delivering immunoglobulins and other compounds transclerally to treat disorders associated with, for example, the retina and choroid (Ambati et al. (2000) supra).

The invention provides a miniaturized, low-profile, implantable, transscleral drug delivery device capable of delivering one or mote drugs at defined rates to a particular target location over a prolonged period of time. The devices of the invention can be used to deliver a drug of interest into a recipient, for example, a mammal, more specifically, a human. In view of its small size, it is contemplated that the drug delivery device may be implanted using minimally invasive procedures into a small body cavity. For example, the device, when attached to a scleral surface, can be accommodated by the eye socket. Thereafter, the device deposits drug onto the scleral surface over a prolonged period of time. The drug then diffuses through the sclera and into the target tissue to ameliorate the symptoms of an ocular disorder and otherwise impart a localized prophylactic and/or therapeutic effect.

Drug Delivery Device

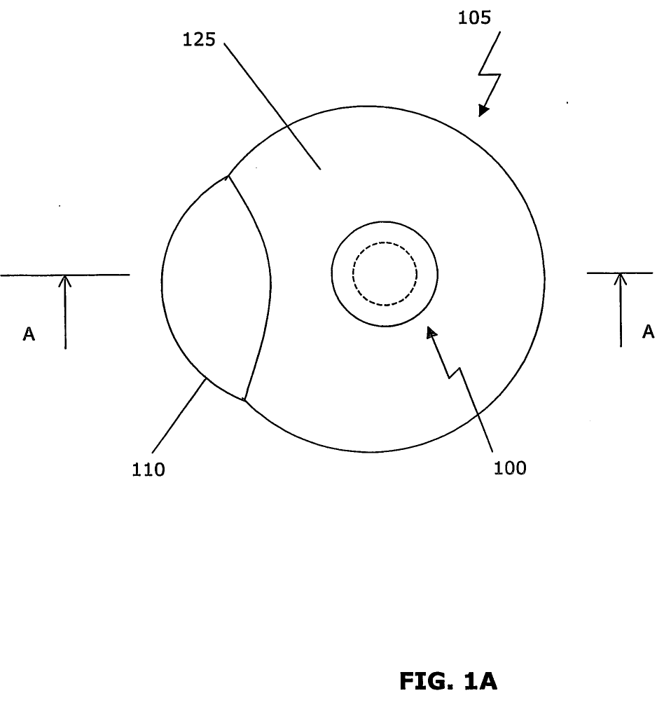

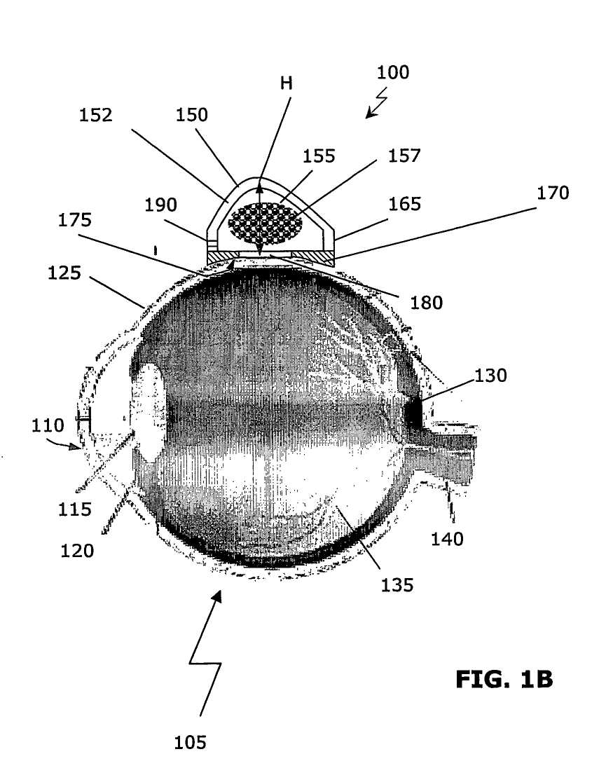

The miniaturized drug delivery device of the invention may be more fully understood by reference to the drawings. Referring to FIGS. 1A-1B, a transscleral drug delivery device 100 according to one embodiment of the invention is attached to an exterior scleral surface of eye 105. The eyeball is shown schematically and in just enough detail to enable an understanding of the present invention. Certain parts of the eye are thus briefly identified with reference numerals. Schematically represented in either or both of FIGS. 1A-1B are cornea 110, lens 115, iris 120, sclera 125, retina 130, vitreal cavity 135, and optic nerve 140.

Still referring to FIG. 1B, the transscleral drug delivery device 100 includes a dome member 150 having a wall 152 and defining a chamber or a cavity 155 for receiving and storing a drug 157. The cavity 155 is in fluid flow communication with the exterior of the dome member 150, so that when the device 100 is attached to the scleral surface 125, the cavity 155 is in fluid flow communication with the sclera 125. The dome member 150 preferably is pre-formed of rigid or semi-rigid material to have a generally outwardly concave shape and a low profile so as to fit easily and closely against eye 105 during the implantation procedure. Other shapes, including shapes having variable curvature, are also contemplated.

Because the device of the invention is designed for implantation into a body and to the extent that the cavity 155 of the dome member 150 is accessible to body fluid, the choice of material for fabricating the dome member 150 and the fluid contacting surface of the inner components of the device 100 is important. Specifically, the tissue and/or body fluid contacting portions of the drug delivery device 100 preferably are fabricated from an inert, biocompatible material. If the tissue and/or body fluid contacting portions of the device are not fabricated from biocompatible materials, then they preferably are encapsulated within a biocompatible material, such as, polyethyleneglycol, polyvinylchloride, polycarbonate, polysulfone, polytetrafluoroethylene, parylene, titanium or the like, prior to implantation.

In addition to biocompatibility, weight, strength, particularly strength-to-thickness ratio, as well as fluid impermeability are other important considerations in the choice of materials. Useful biocompatible materials include, for example, a metal or an alloy of two or more metals, for example, gold, titanium, titanium alloy (such as an alloy including 6% aluminum and 4% vanadium with balance titanium), nickel titanium, stainless steel, anodized aluminum, or a rigid or semi-rigid non-metal, for example, a polymeric composition.

In some embodiments, the material of the device 100 is non-biodegradable so that the device 100 remains implanted in the patient's body substantially indefinitely. In other embodiments, the material of the device 100 is biodegradable after a substantially predetermined period of time, such as, for example, approximately one year. In a particular embodiment, the material of the device 100 is selected such that the device 100 would harmlessly dissolve in the patient's body shortly after the drug delivery process is complete and the disease state resolved.

In some embodiments, the dome member 150 is fabricated with a homopolymer, a copolymer, straight, branched, cross-linked, or a blend thereof that may or may not be biodegradable. Examples of polymers suitable for use in said polymeric composition include silicone, polyvinyl alcohol, polyethylene, polypropylene, nylon, polydimethylsiloxane, polymethyl methacrylate (PMMA), polyurethane, ethylene vinyl acetate, polylactic acid, polycarbonate, cellulose, cellulose acetate, polyglycolic acid, polylactic-glycolic acid, cellulose esters, polyethersulfone, acrylics, their derivatives, and combinations thereof. Examples of suitable soft acrylics are more fully disclosed in U.S. Pat. No. 5,403,901. Further, examples of biodegradable polymers suitable for use with the invention include polyesters composed of homopolymers or copolymers of glycolide and lactide, such as poly(DL-lactic-co-glycolic acid)(“PLGA”), as well as polycaprolactone homopolymers and copolymers.

The polymeric composition may also comprise other conventional materials that affect its physical properties, including, but not limited to, porosity, tortuosity, permeability, rigidity, hardness, and smoothness. Exemplary materials affecting certain ones of these physical properties include conventional plasticizers, filers, and lubricants. The polymeric composition may comprise other conventional materials that affect its chemical properties, including, but not limited to, toxicity and hydrophobicity.

In various embodiments, the dome member 150 fabricated from the polymeric composition may be made by conventional polymer processing methods, including, but not limited to, injection molding, extrusion molding, transfer molding, compression molding, and stereolithography. In one embodiment, the dome member 150 is formed using conventional injection molding techniques. Extrusion or blow molding techniques can also be used. In other embodiments, the dome member 150 fabricated from a metal or a metal alloy can be manufactured by any method or combination of methods known in the art, including, for example, forging, stamping, die casting, thixomolding, machining, turning, sintering, or stereolithography.

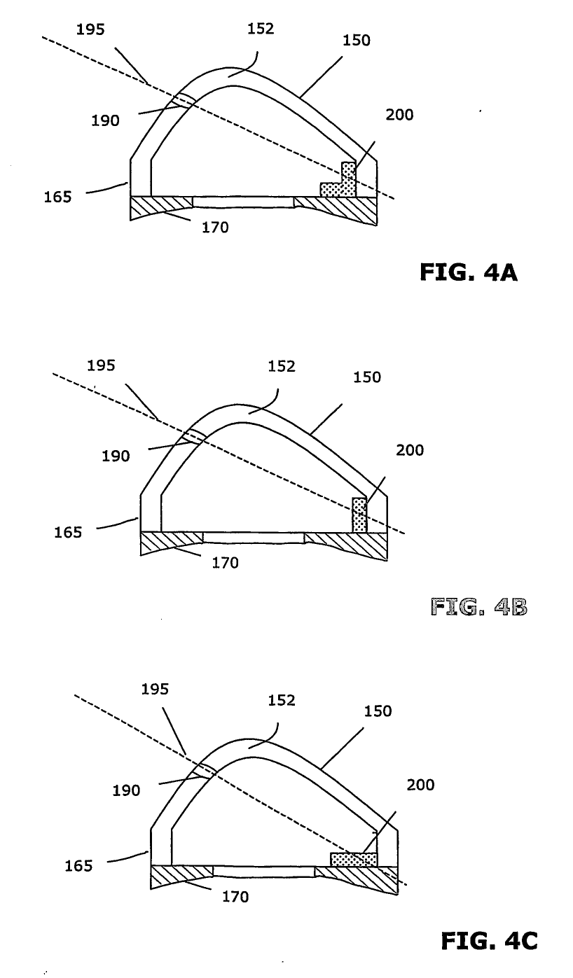

In some embodiments, the material of the dome member 150 is impenetrable by an injection needle or syringe. In other embodiments, an additional structure, such as a puncture guard described in more detail below with reference to FIGS. 4A-4C, is provided to prevent the injection needle from inadvertently contacting the sclera 125.

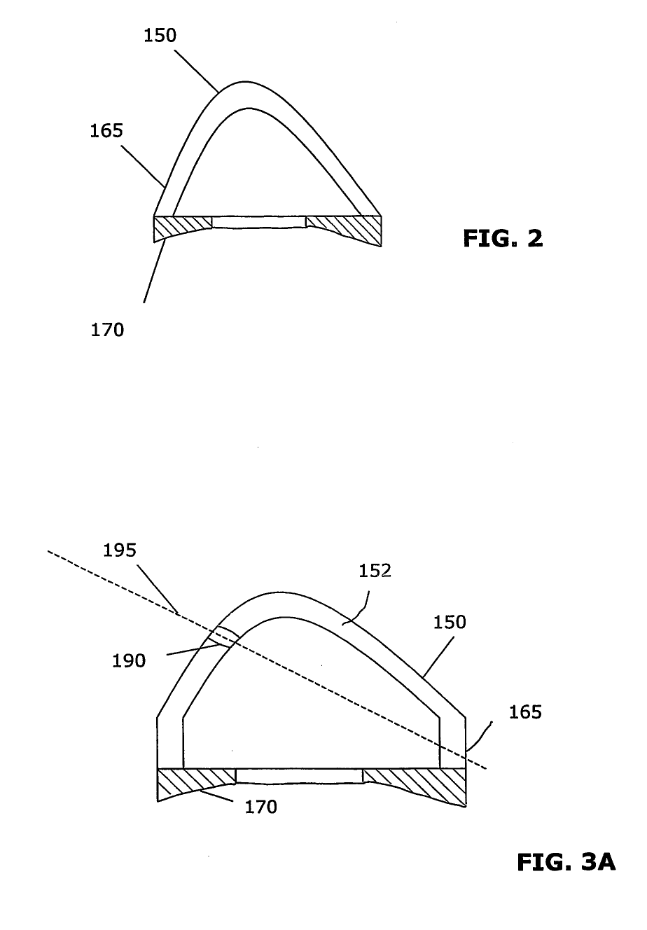

The wall 152 of dome member 150 includes a base portion or region 165 disposed proximate to eye 105 following implantation of the device 100 onto the sclera 125. In various embodiments, the dome member's profile in the base region 165 differs from the profile of the rest of the dome member 150. The transition between profiles is preferably smooth so as to reduce patient's discomfort. As shown in FIG. 1B, in one embodiment of the invention, the base region 165 has a generally tubular shape, that is, a cross-section of the dome member 150 taken parallel to the sclera 125, that remains constant throughout the base region 165. In another embodiment, as shown in FIG. 2, there is no profile variation between the base region 165 and the rest of the dome member 150. In some embodiments, the base region 165 is a separate structure joined in a fluid-tight manner to the dome member 150, by soldering or adhesive bonding.

With continued reference to FIGS. 1A-1B, in one embodiment, base region 165 has a generally circular footprint over the sclera 125. As understood by those skilled in the art, the shape of the footprint may be varied to facilitate implantation. For example, in some embodiments, the base region 165 may have a rounded rectangular, oval, or irregularly-shaped, rounded footprint.

In various embodiments of the invention, the transscleral drug delivery device 100 also includes a base member, for example, a base plate 170 having a scleral-contacting surface 175 of outwardly concave shape or curvature generally complementary to the curvature of the sclera 125. In one embodiment, base plate 170 is an integral part of the dome member 150, such that base plate 170 and dome member 150 are fabricated as a one-piece structure. In other embodiments, the base plate 170 is a separate structure joined in a fluid-tight manner to the base region 165 of the dome member 150, by, for example, soldering or adhesive bonding. In some embodiments, the base plate 170 is made of a tough material impenetrable by an injection needle or syringe, for example, fabricated of a plastic, such as nylon, Kevlar, or polymethyl methacrylate (PMMA), or metal, such as titanium or tantalum. In these embodiments, the base plate 170 may be fabricated from the same material as the dome member 150, or a different material. In other embodiments, an additional structure, such as a puncture guard described in more detail below with reference to FIGS. 4A-4C, is provided to prevent the injection needle from contacting the sclera 125.

In various embodiments, the base region 165 of the dome member 150 or the base plate 170 may optionally define one or more apertures, fenestrations or eyelets to permit the device 100 to be immobilized to the tissue of interest, for example, via sutures or the like. Furthermore, the base region 165 of the dome member 150 may optionally comprise a rim or flange disposed about the circumference as part of or adjacent to base plate 170 to assist in attaching the device 100 to the tissue of interest. In some embodiments, the device 100 is attached onto the eye by affixing the base plate 170 to the sclera 125, by, for example, sutures, passing through eyelets attached to base plate 170 or base region 165, or mattress sutures criss-crossing the dome member 150. Furthermore, the device may be attached to sclera 125 via a biocompatible, non-biodegradable adhesives, such as, for example, a fibrin sealant or other kind of tissue glue. In addition, the base of the device preferably is configured and/or attached to the surface of the eye so that the base is sealed to prevent drug released from the cavity 155 from contacting portions of the scleral surface that are not underneath the base plate 170. In other words, the base region 165 of the device is sealed to prevent drug from leaking out from under the base region 165. The sealing can be accomplished during attachment by applying a biocompatible glue or sealant to the base of the device prior to attachment to the sclera. Alternatively, the base plate may be sealed after attachment of the device by applying a biocompatible glue or sealant around the exterior of the base plate 170 in contact with the sclera 125.

When in use, the device 100 is substantially impermeable to both the body fluids of the environment and to the drug, except through the drug outlet port, and an optional drug inlet port (described in detail below). Referring still to FIG. 1B, in one embodiment, the base plate 170 defines at least one drug outlet port, such as an aperture 180, for maintaining the cavity 155 of the dome member 150 in fluid flow communication with the exterior of the device 100, thus permitting the drug contained with the cavity of the implanted device 100 to exit the device and contact the sclera 125.

The number, configuration, shape, and size of the apertures are chosen to provide the release rate required suiting a treatment regimen. In some embodiments, more than one aperture may be provided in the device for the release of drug. When more than one aperture is provided, the plurality of apertures should be construed to be of functionally equivalent to a single aperture.

As mentioned above, the device 100 is configured to deliver drugs applied to the sclera into the vitreal cavity of the eye over a prolonged period of time. Specifically, it is contemplated that the drug 157 exiting the device 100 diffuses through the sclera 125 and into the target tissue, for example, a vitreal cavity, to ameliorate the symptoms of an ocular disorder and otherwise impart a localized prophylactic and/or therapeutic effect. It is, therefore, desirable that the rate of release of the drug from the device maintains the drug delivered to the sclera in sufficient concentrations so that the drug penetrates through the sclera and into the vitreal cavity in therapeutically effective concentrations. During operation of the device, the sclera 125 in the area either beneath the device 100 or otherwise in fluid communication with the chamber 155 is not punctured or made more permeable by permeability enhancing agents. Instead, the therapeutically effective concentration is achieved by selecting a suitable rate of release of the drug 157, which, in Which, is achieved by providing an aperture of proper area relative to the area of the device 100 and taking into account parameters, such as the solubility properties of the drug 157.

Consistent with the considerations mentioned above, in various embodiments, the total area of the aperture exceeds 25%, for example, ranges from 25% to 50%, of the footprint of the base region 165 over the base plate 170. In a particular embodiment, the base region 165 has a circular footprint over the base plate 170 having a first diameter. The base plate 170 defines a circular aperture 180 having a second diameter that equals at least one half of the first diameter.

The aperture 180 may be made in the base plate 170 using a needle or other form of boring instrument such as a mechanical drill or a laser to remove a section of the base plate 170. Alternatively, a specially designed punch tip may be incorporated into the compressing equipment, in order to pierce through the base plate 170 at the point of compaction.

The chamber 155 has a maximum height dimension indicated by the numeral H. As a non-limiting example, this maximum height dimension ranges between about 3 mm and about 7 mm, for example, is about 4 mm. It is contemplated that the length and width dimensions of the cavity 155, measured generally spherically of the wall of the dome member 150, are relatively much greater than the maximum height H. For the embodiment shown in FIG. 1B, but again not limiting to the invention, the footprint of the base region 165 ranges from about 25 mm² to about 400 mm², for example, totals approximately 300 mm². In certain embodiments, the drug outlet part has a surface area at least 25% of the footprint of the base region. For example, when the base region is circular and has a diameter in the range from 5 mm to 25 mm, the diameter of the drug aperture part is in the range from 2.5 mm to 12.5 mm. However, it is contemplated that the base region and the drug outlet part can have a variety of different configurations but yet the surface area of the drug outlet part is greater than 25% of the surface area of the base region.

Preferably, the volume of the chamber 155 is such that the device 100 holds sufficient amount of the drug to provide a continuous delivery over the extended delivery period, e.g., several weeks, months, or even longer. The volume needed thus depends on characteristics such as drug solubility, drug delivery rate, period of delivery, drug's half life, etc. Once implanted, the device continuously delivers the drug to vitreal cavity of the eye for prolonged period of time until replenishment.

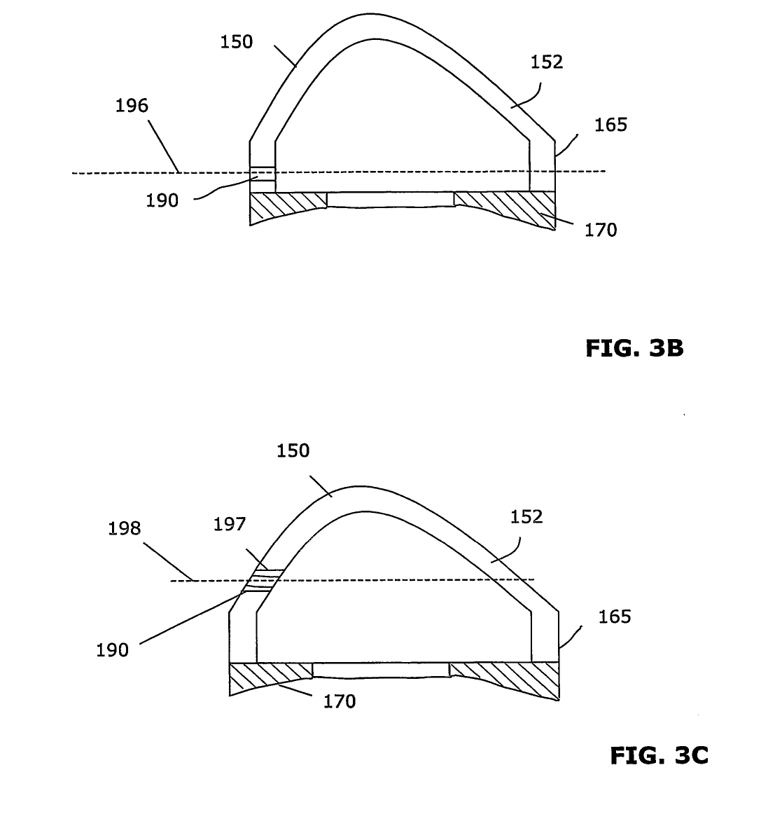

In order to provide for replenishment of the drug in situ without surgery or other invasive procedure, the device 100 includes a drug inlet port 190 for injecting drug 157 into cavity 155 of the implanted device 100. In various embodiments, the drug inlet port 190 is an aperture defined by wall 152 of the dome member 150, as shown in FIGS. 3A-3B. As discussed above, it is desirable to prevent inadvertent puncture of the eyeball by an injection needle used to replenish the supply of drug in the device 100. Towards that end, the drug inlet port 190 is configured to minimize the possibility of the needle contacting the sclera 125. Also, in various embodiments, the drug inlet port 190, may also include a filler material, such as, for example, polydimethylsiloxane or other kinds of silicone rubber, which is penetrable by a needle or syringe but which reseals itself when the needle is withdrawn so that the port is normally fluid-impervious. The filler material can be colored to provide a marker or target which is visible exteriorly, especially through covering tissue or patches, to facilitate location of the port by attending medical personnel.

Referring to FIG. 3A, in various embodiments, the drug inlet port 190 is an aperture defined by the wall 152 of the dome member 150. The location of the aperture is selected such that an axis 195 perpendicular to the aperture 190 does not intersect the base plate 170, thereby minimizing the possibility of contacting the sclera 125. For example, as shown in FIG. 3B, in a particular embodiment, the dome member 150 includes the base region 165 having a generally tubular shape, as shown in FIG. 1B. Drug inlet port 190 is an aperture defined by the wall 152 of the dome member 150 in the base region 165. In this embodiment, the wall 152 of the dome member 150 is substantially perpendicular to the base plate 170 and the scleral surface in the area of implantation of the device 100. As a result, axis 196 perpendicular to the aperture 190 is generally parallel to the base plate 170 and the scleral surface in the area of implantation of the device 100, and, therefore, a needle inserted through aperture substantially perpendicular thereto will not contact the scleral surface of the eye.

Referring to FIG. 3C, in other embodiments, the drug inlet port 190 further includes a generally tubular member 197 that is disposed in an aperture of the drug inlet port 190 defined by the dome member. In one embodiment, the tubular member 197 is a separate structure that is adhesively attached within the aperture. In another embodiment, the tubular member 197 is fabricated as an integral part of the dome member 150. The tubular member 197 defines a lumen having a central longitudinal axis 198. The central longitudinal axis 198 of the lumen does not intersect the base plate, for example, in one embodiment, is substantially parallel to the base plate. The tubular member 197, therefore, serves as a guide directing a needle inserted through the drug inlet port 190 so that it would not contact the sclera 125. For example, the tubular member may guide the needle either parallel to, as mentioned above, or extending away from the base plate 170. Because the orientation of the tubular member 197 in the drug inlet port 190 in relation to the base plate 170 may be chosen substantially arbitrarily, direction of the central longitudinal axis 198 may deviate from the direction of the axis perpendicular to the aperture of the drug inlet port 198. In this embodiment, a choice for the safe location of the drug inlet port 190 in the wall of the dome member is less constrained compared to the embodiments of FIGS. 3A-3B.

Referring now to FIGS. 4A-4C, to further minimize a possibility of inadvertent puncture of the eyeball by an injection needle used to replenish the supply of drugs or other agents, the device 100 optionally includes a puncture guard 200 disposed adjacent to at least one surface of the base plate, or at least one surface of the dome member. The location for the puncture guard 200 is selected to prevent an injection needle inserted through the drug inlet port 190 or through the wall 152 of the dome member 150 from contacting the sclera 125

In some embodiments, the puncture guard 200 is a separate shield structure attached to a portion of at least one surface of the base plate, or at least one surface of the dome member. The puncture guard 200 can be attached by soldering or adhesive bonding. In this embodiment, the puncture guard 200 is fabricated from a tough material impenetrable by an injection needle or syringe, for example, a plastic, such as nylon, Kevlar, or PMMA, or metal, such as titanium or tantalum, or other metal or metal alloys mentioned above as suitable materials for the dome member 150. In other embodiments, the puncture guard 200 is an integral part of the wall 152 of the dome member 150 where the material of the dome member 150 is selected to be needle-impenetrable.

Referring to FIG. 4A, in one embodiment, the puncture guard 200 is a L-shaped shield disposed on the inside surface of the dome member 150 at the junction of the base region 165 of the dome member 150 and the base plate 170 substantially opposite the drug inlet port 190. Other shapes of the puncture guard 200, for example, a funnel, are also contemplated. In some embodiments, the puncture guard 200 is a plate disposed on inside surface of either the base region 165 or the base plate 170 of the dome member 150, as shown in FIGS. 4B-4C, respectively. The puncture guard 200 may also be disposed on the outside surfaces of either the base region 165 or the base plate 170 (not shown).

The method of the present invention and use of the device 100 are best described by reference to FIGS. 1A-1B. In one embodiment, the device 100 is implanted within the orbital socket. In one procedure, device 100 is placed under the conjunctiva and Tenon's capsule, so that it is located between the superior and lateral rectus muscles and slightly posteriorly of the equator of the eyeball. When located as such, the drug inlet port 190 faces anteriorly.

A supply of drug 157 is placed in the cavity 155 before or after implantation. Examples of drugs that may be used with the device 100 are discussed in more detail below. If drugs or other agents need to be injected after the device 100 is implanted, the eyelid is lifted and the eye is rotated to expose the region where the device 100 is implanted. The drug inlet port 190, when exposed, can be penetrated with an injection needle of a syringe (not shown) to introduce drug 157 into the cavity 155.

If a large volume of drug 157 is to be introduced into cavity 155, either initially or to refill the device 100 at a later date, venting of the cavity 155 by a second needle (not shown but placed through the injection port 190 simultaneously with injection) may be required. Injection of small volumes of drug 157 into the cavity 155, however, may not require venting.

Drug and Drug Formulation

As discussed above, it is understood that the drug delivery device of the invention can be used to deliver one or more drugs to a particular target site, specifically, to the scleral surface of an eye. When attached, the device delivers drug to the surface of the eye, which then passes through the sclera and into the target tissue to ameliorate the symptoms of an ocular disorder.

The drug 157 can be disposed within the cavity 155 of the device 100 in solid, liquid, or gel form. As used herein, the term “drug” is understood to mean any natural or synthetic, organic or inorganic, physiologically or pharmacologically active substance capable of producing a localized or systemic prophylactic and/or therapeutic effect when administered to an animal. A drug includes (i) any active drug, (ii) any drug precursor or pro-drug that may be metabolized within the animal to produce an active drug, (iii) combinations of drugs, (iv) combinations of drug precursors, (v) combinations of a drug with a drug precursor, and (vi) any of the foregoing in combination with a pharmaceutically acceptable carrier, excipient or formulating agent.

The drug may include, for example, a protein (for example, an antibody or an antigen binding portion thereof), a polypeptide, a nucleic acid (for example, deoxyribonucleic acid and/or ribonucleic acid), a peptidyl nucleic acid, a polysaccharide, a fatty acid (for example, prostaglandin), an organic molecule and an inorganic molecule, that has prophylactic and/or therapeutic value, i.e., elicits a desired effect, when administered to an animal. The drug can include, for example, a hormone or synthetic hormone, an anti-infective agent (for example, an antibiotic, an anti-viral agent, and an anti-fungal agent), a chemotherapeutic agent (for example, methotrexate, chlorambucil, cyclosporine, and interferon), an autonomic drug (for example, an anticholinergic agent, adrenergic agent, adrenergic blocking agent, and a skeletal muscle relaxant), a blood formation or blood coagulation modulating agent (for example, an anti-anemia drug, coagulant and an anti-coagulant, hemorrhagic agent, and a thrombolytic agent), a cardiovascular drug (for example, a hypotensive agent, vasodilating agent, inotropic agent, β-blocker, and a sclerosing agent), a central nervous system agent (for example, an analgesic, an antipyretic, and an anti-convulsant), an immunomodulating agent (for example, etanercept, or an immunosuppresant), an anti-inflammatory agent (for example, a steroid, and interferon α), an anti-obesity agent (for example, leptin), an anti-lipemic agent (for example, an inhibitor of hydroxymethylglutaryl co-enzyme A reductase), an anti-emetic agent (for example, cisapride and metoclopramide), an anti-migraine medication (for example, imitrex), a chelating agent (for example, the iron chelator desferoxamine), and a contraceptive or fertility agent.

The drug also embraces an angiogenesis inhibitor, i.e., a compound that reduces or inhibits the formation of new blood vessels in a mammal. Angiogenesis inhibitors may be useful in the treatment of various disorders associated with neovascularization, for example, certain ocular disorders associated with neovascularization. Examples of useful angiogenesis inhibitors, include, for example, protein/peptide inhibitors of angiogenesis such as: angiostatin, a proteolytic fragment of plasminogen (O'Reilly et al. (1994) CELL 79: 315-328, and U.S. Pat. Nos. 5,733,876; 5,837,682; and 5,885,795) including full length amino acid sequences of angiostatin, bioactive fragments thereof, and analogs thereof; endostatin, a proteolytic fragment of collagen XVIII (O'Reilly et al. (1997) CELL 88: 277-285, Cirri et al. (1999) INT. BIOL. MARKER 14: 263-267, and U.S. Pat. No. 5,854,205) including full length amino acid sequences of endostatin, bioactive fragments thereof, and analogs thereof; peptides containing the RGD tripeptide sequence and capable of binding the α-_{V}β₃ integrin (Brooks et al. (1994) C_{ELL} 79: 1157-1164, Brooks et al. (1994) SCIENCE 264: 569-571); certain antibodies and antigen binding fragments thereof and peptides that bind preferentially to the α-ᵥβ₃ integrin found on tumor vascular epithelial cells (Brooks et al., supra, Friedlander et al. (1996) PROC. NATL. A_{CAD.} S_{CI.} USA 93: 9764-9769); certain antibodies and antigen binding fragments thereof and peptides that bind preferentially to and block or reduce the binding activity of the Epidermal Growth Factor receptor (Ciardiello et al. (1996) J. NATL. CANCER INST. 88: 1770-1776, Ciardiello et al. (2000) CLIN. CANCER RES. 6:3739-3747); antibodies, proteins, peptides and/or nucleic acids that preferentially bind to and inhibit or reduce the activity of Vascular Endothelial Growth Factor (VEGF) (Adamis et al. (1996) ARCH OPTHALMOL 114:66-71), antibodies, proteins, and/or peptides that bind preferentially to and block or reduce the binding activity of Vascular Endothelial Growth Factor receptor; anti-Fibroblast Growth Factor, anti-Epidermal Growth Factor (Ciardiello et al. (2000) CLIN. CANCER RES. 6: 3739-3747) including full length amino acid sequences, bioactive fragments and analogs thereof, and Pigment Epithelium-derived Growth Factor (Dawson (1999) SCIENCE 2035: 245-248) including full length amino acid sequences, bioactive fragments and analogs thereof Bioactive fragments refer to portions of the intact protein that have at least 30%, more preferably at least 70%, and most preferably at least 90% of the biological activity of the intact proteins. Analogs refer to species and allelic variants of the intact protein, or amino acid replacements, insertions or deletions thereof that have at least 30%, more preferably at least 70%, and most preferably 90% of the biological activity of the intact protein.

Other angiogenesis inhibitors include, for example, COX-2 selective inhibitors (Masferrer et al. (1998) PROC. AMER. ASSOC. CANCER RES. 39: 271; Ershov et al. (1999) J. NEUROSCI. RES. 15: 254-261; Masferrer et al. (2000) CURR. MED. CHEM. 7: 1163-1170); tyrosine kinase inhibitors, for example, PD 173074 (Dimitroff et al. (1999) INVEST. NEW DRUGS 17: 121-135), halofuginone (Abramovitch et al. (1999) NEOPLASIA 1: 321-329; Elkin et al. (1999) CANCER RES. 5: 1982-1988), AGM-1470 (Brem et al. (1993) J. PED. SURGERY 28: 1253-1257), angiogenic steroids, for example, hydrocortisone and anecortave acetate (Penn et al. (2000) INVEST. OPHTHALMOL. VIS. SCI. 42: 283-290), thrombospondin-1 (Shafiee et al. (2000) INVEST. OPHTHALMOL. VIS. SCI. 8: 2378-2388; Nor et al. (2000) J. VASC. RES. 37: 09-218), UCN-01 (Kruger et al. (1998-1999) INVASION METASTASIS 18: 209-218), CM101 (Sundell et al. (1997) CLIN. CANCER RES. 3: 365-372); fumagillin and analogues such as AGM-1470 (Ingber et al. (1990) NATURE 348: 555-557), and other small molecules such as thalidomide (D'Amato et al. (1994) PROC. NATL. ACAD. SCI. USA 91: 4082-4085).

Several cytokines including bioactive fragments thereof and analogs thereof have also been reported to have anti-angiogenic activity and thus may be delivered using the device of the invention. Examples include, for example, IL-12, which reportedly works through an IFN-γ-dependent mechanism (Voest et al. (1995) J. NATL. CANC. INST. 87: 581-586); IFN-α, which has been shown to be anti-angiogenic alone or in combination with other inhibitors (Brem et al. (1993) J. PEDIATR. SURG. 28: 1253-1257). Furthermore, the interferons IFN-α, IFN-β and IFN-γ reportedly have immunological effects, as well as anti-angiogenic properties, that are independent of their anti-viral activities.

The drugs suitable for use with the invention also embrace neuroprotective agents, i.e., agents capable of retarding, reducing or minimizing the death of neuronal cells. Neuroprotective agents may be useful in the treatment of various disorders associated with neuronal cell death, for example, certain ocular disorders including, for example, macular degeneration, retinitis pigmentosa, glaucoma and diabetic retinopathy. Examples of neuroprotective agents include, for example, apoptosis inhibitors, for example, neurotrophic factors, cAMP elevating agents, and caspase inhibitors.

Exemplary neurotrophic factors include, for example, Brain Derived Growth Factor and bioactive fragments and analogs thereof (Caffe et al. (2001) INVEST OPHTHATMOL VIS SCI. 42: 275-82); Fibroblast Growth Factor and bioactive fragments and analogs thereof (Bryckaert et al. (1999) ONCOGENE 18: 7584-7593); Pigment Epithelium Derived Growth Factor and bioactive fragments and analogs thereof; and Insulin-like Growth Factors (IGF) and bioactive fragments and analogs thereof, for example, IGF-I and IGF-II (Rukenstein et al. (1991) J. NEUROSCI. 11: 552-2563) and cytokine-associated neurotrophic factors. Exemplary cAMP elevating agents include, for example, 8-(4-chlorophenylthio)-adenosine-3′:5′-cyclic-monophosphate (CPT-cAMP) (Koike (1992) PROG. NEURO-PSYCHOPHARMACOL AND BIOL. PSYCHIAT. 16: 95-106), forskolin, isobutyl methylxanthine, cholera toxin (Martin et al. (1992) J. NEUROBIOL 23: 1205-1220), 8-bromo-cAMP, N⁶, O^{2′}-dibutyryl-cAMP and N⁶, O^{2′}dioctanoyl-cAMP (Rydel and Greene (1988) PROC. NAT'L. ACAD. SCI. USA 85: 1257-1261). Exemplary caspase inhibitors include, for example, caspase-1 inhibitors, for example, Ac—N-Me-Tyr-Val-Ala-Asp-aldehyde, caspase-2 inhibitors, for example, Ac-Val-Asp-Val-Ala-Asp-aldehyde, caspase-3 inhibitors, for example, Ac-Asp-Glu-Val-Asp-aldehyde, caspase-4 inhibitors, for example, Ac-Leu-Glu-Val-Asp-aldehyde, caspase-6 inhibitors, for example, Ac-Val-Glu-Ile-Asp-aldehyde, caspase-8 inhibitors, for example, Ac-Asp-Glu-Val-Asp-aldehyde, and caspase-9 inhibitors, for example, Ac-Asp-Glu-Val-Asp-aldehyde, each of which can be obtained from Bachem Bioscience Inc., Pa.

As discussed, the device of the invention is useful in the treatment of a variety of ocular disorders, such as diabetic retinopathy, glaucoma, macular degeneration, neovascularization, inflammation of retina, macular edema, conjunctivitis, and others. For example, the drug delivery device may deliver an anti-infective agent, such as, an antibiotic, anti-viral agent or anti-fungal agent, for the treatment of an ocular infection. Similarly, the device may deliver a steroid, for example, hydrocortisone, dexamethasone sodium phosphate or methylprednisolone acetate, for the treatment of an inflammatory disease of the eye. The device may be used to deliver a chemotherapeutic or cytotoxic agent, for example, methotrexate, chlorambucil, cyclosporine, or interferon, for the treatment of an ocular neoplasm. Furthermore, the device may be useful in delivering one or more drugs for the treatment of certain degenerative ocular disorders, for example, (i) an adrenergic agonist, such as, epinephrine (Epifrin), dipivefrin (Propine), apraclonidine (Iopidine), or brimonidine (Alphgan); a β-blocker, such as, betaxolol (Betoptic) or timolol (Timoptic); a carbonic anhydrase inhibitor, such as, acetazolamide (Diamox), methazolamide (Neptazane), dorzolamide (Trusopt), or brinzolamide (Azopt); prostglandin analogues, such as, latanoprost (Xalatan), for the treatment of glaucoma, (ii) an integrin (such as, a lymphocyte function associated molecule (LFA-1), Mac-1 or p150,95) antagonist; a selectin (such as, E-selectin, P-selectin and L-selectin) antagonist; an adhesion molecule (such as, an intercellular Adhesion molecule (ICAM)-1, ICAM-2, ICAM-3) antagonist; a Platelet Endothelial Adhesion Molecule antagonist; a Vascular Cell Adhesion Molecule antagonist; a leukocyte adhesion inducing cytokine or growth factor (such as, Tumor Necrosis Factor-α, or Interleukin-1β) antagonist; a Monocyte Chemotactic Protein-1 antagonist; a VEGF antagonist, and other molecules described in PCT/US99/31215 for the treatment of diabetic retinopathy, (iii) an anti-inflammatory drug, such as, a steroid (for example, hydrocortisone, dexamethasone sodium phosphate or methylprednisolone acetate), indomethacin, naprosyn, or a VEGF antagonist for the treatment of macular edema secondary to certain retinal vascular disorders. As used herein, the antagonist may comprise, without limitation, an antibody, an antigen binding portion thereof or a biosynthetic antibody binding site that binds a particular target protein, for example, ICAM-1; an antisense molecule that hybridizes in vivo to a nucleic acid encoding a target protein or a regulatory element associated therewith, or a ribozyme, aptamer, or small molecule that binds to and/or inhibits a target protein, for example, ICAM-1, or that binds to and/or inhibits, reduces or otherwise modulates expression of nucleic acid encoding a target protein, for example, ICAM-1.

The drug or drugs of interest may be introduced into cavity 155 either in pure form or as a formulation, for example, in combination with a pharmaceutically acceptable carrier or encapsulated within a release system. A release system can include a matrix of a biodegradable material or a material which releases incorporated drug by diffusion. The drugs can be homogeneously or heterogeneously distributed within the release system. A variety of release systems may be useful in the practice of the invention, however, the choice of the appropriate system will depend upon rate of drug release required by a particular drug regime. Both non-degradable and degradable release systems can be used. Suitable release systems include polymers and polymeric matrices, non-polymeric matrices, or inorganic and organic excipients and diluents such as, but not limited to, calcium carbonate and sugar. Release systems may be natural or synthetic. However, synthetic release systems are preferred because generally they are more reliable, more reproducible and produce more defined release profiles. The release system material can be selected so that drugs having different molecular weights are released from a particular cavity by diffusion through or degradation of the material. Biodegradable polymers, bioerodible hydrogels, and protein delivery systems currently are preferred for drug release via diffusion or degradation.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

In one embodiment of the invention, the device 100 contains an aptamer, preferably an anti-Vascular Endothelial Growth Factor (VEGF) aptamer, optionally encapsulated in biocompatible polymer microspheres. The aptamers, such as the anti-VEGF aptamers, may be used in the treatment of a variety of disorders associated with VEGF activity, for example, neovasculature associated with the activation of the VEGF receptor by a VEGF molecule. In such a system, the administration of the VEGF aptamer acts by binding the VEGF receptor to block, prevent or otherwise minimize the binding of a naturally occurring VEGF molecule to that receptor. The aptamers may be useful in the treatment of ocular disorders that are initiated, mediated, or facilitated by means of the VEGF receptor.

In the case of aptamer containing microspheres, the microspheres may deliver the aptamer of interest over a prolonged period of time into the tissue or body fluid surrounding the microspheres thereby imparting a localized prophylactic and/or therapeutic effect. It is contemplated that the microspheres may administer the aptamer of interest over a period of weeks (for example, 1, 2, or 3 weeks), and more preferably months (for example, 1, 2, 3, 4, 5, 6, 7, 8, 9, 10, or 11 months), or longer.

The VEGF aptamer can be released from the microspheres under physiological conditions over a period of time, typically at least 20 days, and, when released, retains its biological activity. The microspheres include the anti-VEGF aptamer and a biocompatible polymer, where the amount of the aptamer in the microsphere varies from 0.1% to 30% (w/w), 0.1% to 10% (w/w), or, desirably, 0.5% to 5% (w/w) of the microsphere. The microspheres may further include a stabilizer, for example, a sugar, for example, trehalose. In one embodiment, the mass ratio of aptamer to trehalose in the microsphere is at least 1:3.

In some embodiments, the biocompatible polymer is a degradable polymer. Degradable polymers useful in the preparation of the microspheres indude polycarbonates, polyanhydrides, polyamides, polyesters, polyorthoesters, and copolymers or mixtures thereof. Exemplary polyesters include poly(lactic acid), poly(glycolic add), poly(lactic acid-co-glycolic acid), polycaprolactone, blends thereof and copolymers thereof. Desirably, the half-life for the degradation of the degradable polymer under physiological conditions is at least about 20 days and more preferably is at least about 30 days. In a preferred embodiment, the microspheres comprise a poly(lactic acid co-glycolic acid) (PLGA) polymer.

In other embodiments, the biocompatible polymer is a non-degradable polymer. Non-degradable polymers useful in the preparation of the microspheres include polyethers, vinyl polymers, polyurethanes, cellulose-based polymers, and polysiloxanes. Exemplary polyethers include poly(ethylene oxide), poly(ethylene glycol), and poly(tetramethylene oxide). Exemplary vinyl polymers include polyacrylates, acrylic acids, poly(vinyl alcohol), poly(vinyl pyrolidone), and poly(vinyl acetate). Exemplary cellulose-based polymers include cellulose, alkyl cellulose, hydroxyalkyl cellulose, cellulose ethers, cellulose esters, nitrocellulose, and cellulose acetates.

Whichever biocompatible polymer is used, in one embodiment, the microspheres preferably have an average diameter in the range from about 1 μm to about 200 μm, from about 5 μm to about 100 μm, and from about 10 μm to about 50 μm. In one embodiment, the microspheres have an average diameter of about 15 μm.

The microspheres may be used to deliver an aptamer of interest to a preselected locus, for example, an eye, in a mammal for example, a human, on a sustained basis. In a preferred embodiment, the microspheres of the invention permit the sustained delivery of an anti-VEGF aptamer. One anti-VEGF aptamer of interest is known in the art as EYE001 and was formerly known in the art as NX1838 (see, Drolet et al. (2000) PHARM. RES. 17:1503-1510; Ruckman et al. (1998) J. BIOL. CHEM. 273:20556-20567; Carrasquillo et al. (2003) INVEST. OPHTHMAL. VIS. SCI. 44:290-299). EYE001 is available from Eyetech Pharmaceuticals (New York, N.Y.) and was identified by the systematic evolution of ligands by exponential enrichment (SELEX) process (Ruckman et al. (1998) J. BIOL. CHEM. 273:20556-20567; Costantino et al. (1998) J. PHARM. SCI. 87:1412-1420). EYE001 can be supplied as a liquid formulation of 3 mg/200 μL saline solution.

EYE001 is a pegylated RNA aptamer of 50 kDa, with an A-type secondary structure, 40 mg/mL solubility, and a net negative charge of −28. The structure of EYE001 is 5′-[40 kd PEG]-[HN—(CH₂)₅O]-pC_{f}pGₘpGₘpAᵣpAᵣpU_{f}pC_{f}pAₘpGₘpU_{f}pGₘpAₘpAₘpU_{f}pGₘpC_{f}pU_{f}pU_{f}pAₘpU_{f}pAₘpC_{f}pAₘpU_{f}pC_{f}pC_{f}pGₘ3′-p-3′dT. The 40 kd PEG component represents two 20 kilodalton-poly(ethylene glycol)polymer chains covalently attached to the two amine groups on a lysine residue via carbamate linkages. This moiety is in turn linked to the oligonucleotide via a bifunctional amino linker, [HN—(CH₂)₅O—]. The linker is attached to the oligonucleotide by a standard phosphodiester bond; p represents the phosphodiester functional groups that link sequential nucleosides and that link the amino linker to the oligonucleotide. All of the phosphodiester groups are negatively charged at neutral pH and have a sodium atom as the counter ion; Gₘ or Aₘ and C_{f} or U_{f} and Aᵣ represent 2′-methoxy, 2′-fluoro and 2′-hydroxy variations of their respective purines and pyrimidines; C, A, U, and G is the single letter code for cytidylic, adenylic, uridylic, and guanylic acids. All phosphodiester linkages of this compound, with the exception of the 3′-terminus, connect the 5′ and 3′ oxygens of the ribose ring. As shown, the phosphodiester linkage between the 3′-terminal dT and the penultimate Gₘ links their respective 3′-oxygens. This is referred to as a 3′,3′ cap.

Although the EYE001 aptamer is preferred, it is contemplated that the microspheres may encapsulate other aptamers of interest and release them on a sustained basis.

In order to permit sustained delivery of an aptamer of interest, the aptamer is encapsulated within a microsphere comprising a biocompatible polymer. The choice of the appropriate microsphere system will depend upon rate of aptamer release required by a particular regime. The aptamer may be homogeneously or heterogeneously distributed within the microspheres. Furthermore, both non-degradable and degradable microspheres can be used. Suitable microspheres may include polymers and polymeric matrices, non-polymeric matrices, or inorganic and organic excipients and diluents such as, but not limited to, calcium carbonate and sugar. Synthetic polymers are preferred because generally they are more reliable, more reproducible and produce more defined release profiles. The microspheres can be designed so that aptamers having different molecular weights are released by diffusion through or degradation of the microspheres.

As mentioned, it is contemplated that useful biocompatible polymers may include biodegradable and/or non-biodegradable polymers. Suitable biodegradable polymers useful in the preparation of the microspheres include polycarbonates, polyanhydrides, polyamides, polyesters, polyorthoesters, and copolymers or mixtures thereof. Exemplary polyesters include poly(lactic acid), poly(glycolic acid), poly(lactic acid-co-glycolic acid), polycaprolactone, blends thereof and copolymers thereof. Desirably, the half-life for the degradation of the degradable polymer under physiological conditions is at least about 20 days and more preferably is at least about 30 days. Suitable non-biodegradable polymers useful in the preparation of microspheres include polyethers, vinyl polymers, polyurethanes, cellulose-based polymers, and polysiloxanes. Exemplary polyethers include poly(ethylene oxide), poly(ethylene glycol), and poly(tetramethylene oxide). Exemplary vinyl polymers include polyacrylates, acrylic acids, poly(vinyl alcohol), poly(vinyl pyrolidone), and poly(vinyl acetate). Exemplary cellulose-based polymers include cellulose, alkyl cellulose, hydroxyalkyl cellulose, cellulose ethers, cellulose esters, nitrocellulose, and cellulose acetates.

It is contemplated that in order to produce the appropriate release kinetics, the microspheres may comprise one or more biodegradable polymers or one or more non-biodegradable polymers. Furthermore, it is contemplated that the microspheres may comprise one or more biodegradable polymers in combination with one or more non-biodegradable polymers. Whichever biocompatible polymer is used, in one embodiment, the microspheres preferably have an average diameter in the range from about 1 μm to about 200 μm, from about 5 μm to about 100 μm, and from about 10 μm to about 50 μm. In one embodiment the microspheres have an average diameter of about 15 μm.

In a particular embodiment, the microspheres are fabricated from PLGA. Aptamer containing PLGA microspheres can be prepared, for example, using non-aqueous oil-in-oil methods (see, Carrasquillo et al. (2001) J. CONTROL RELEASE 76:199-208). Briefly, 25 to 30 mg of solid aptamer is suspended in a solution of 200 mg/2 mL PLGA (Resomer 502 H, i.v. (inherent viscosity) 0.16-0.24 dL/g, 0.10% in chloroform, 25° C., molecular weight [Mw] 10 to 12 kDa, half-life for degradation approximately 1 to 1.5 months; Boehringer Ingelheim Pharma KG, Ingelheim, Germany) in methylene chloride using a homogenizer (Polytron, model PT 1200C; Brinkman, Westbury, N.Y.) having a standard 12-mm diameter generator at approximately 20,000 rpm for 1 minute. After suspension of the aptamer, a coacervating agent, for example, poly(dimethylsiloxane), optionally can be added at a rate of 2 mL/min under constant homogenization, to ensure homogeneous dispersion of the coacervating agent, phase separation of PLGA dissolved in methylene chloride, and formation of microspheres. The coacervating mixture containing the microspheres then is poured into an Erlenmeyer flask containing 50 mL heptane under constant agitation and stirred for 3 hours at room temperature to allow for hardening of the microspheres. Microspheres then are collected by filtration with the use of a 0.22-μm nylon filter, washed twice with heptane, and dried for 24 hours at a vacuum of 80 mbar.

Encapsulation efficiency can be determined using standard methodologies (Carrasquillo et al. (2001) J. PHARM PHARMACOL. 53:115-120). For example, ten milligrams of PLGA microspheres are placed in 2 mL methylene chloride and stirred for 30 minutes to dissolve the polymer. The solution then is centrifuged at 10,000 rpm for 10 minutes to precipitate the insoluble RNA aptamer. The supernatant then is removed, and the remaining methylene chloride allowed to evaporate. In order to ensure evaporation of the methylene chloride, the sample can be placed in a vacuum for 24 hours. The aptamer then is dissolved in Dulbecco's phosphate-buffered saline (DPBS; GibcoBRL, Grand Island, N.Y.), and the concentration of entrapped aptamer in PLGA determined spectrophotometrically. The percentage encapsulation efficiency can be calculated by relating the experimental aptamer entrapment to the theoretical aptamer entrapment: (experimental/theoretical)×100.

In one embodiment, the microspheres include the anti-VEGF aptamer and a biocompatible polymer, where the amount of the aptamer in the microsphere varies from 0.1% to 30% (w/w), 0.1% to 10% (w/w), or, desirably, 0.5% to 5% (w/w) of the microsphere. It is understood that nucleic acids may suffer from depurination and become susceptible to free radical oxidation in aqueous solutions (Lindahl (1993) NATURE 362:709-715; Demple et al. (1994) ANNU REV BIOCHEM. 63:915-948). This effect may be reduced, minimized or eliminated by the addition of a stabilizer, for example, a sugar. An effective stabilizer is the sugar, trehalose. In one embodiment, the mass ratio of aptamer to trehalose in the microsphere is at least 1:3.

It is contemplated that the microspheres may comprise an anti-VEGF aptamer in combination with another angiogenesis inhibitor, that is, a compound that reduces or inhibits the formation of new blood vessels in a mammal. For example, the microspheres may comprise two or more different anti-angiogenesis aptamers. Alternatively, the microspheres in addition to containing an anti-VEGF aptamer may also include another type of angiogenesis inhibitor, for example, an angiogenic steroid, for example, hydrocortisone and anecortave acetate (Penn et al. (2000) INVEST. OPHTHALMOL. VIS. SCI. 42:283-290), or another small molecule, for example, thalidomide D'Amato et al. (1994) PROC. NATL. ACAD. SCI. USA 91:4082-4085).

It is contemplated that the aptamer-containing microspheres delivered to the scleral surface of the eye using the device 100 may be used in a variety of different applications. In one embodiment, the microspheres may be used to administer the aptamers to an eye thereby to treat or ameliorate the symptoms of one or more ocular disorders. For example, the microspheres may be particularly useful in the treatment of a variety of ocular disorders, for example, optic disc neovasculation, iris neovascularization, retinal neovascularization, choroidal neovasculation, corneal neovascularization, vitreal neovascularization, glaucoma, pannus, pterygium, macular edema, vascular retinopathy, retinal degeneration, uveitis, inflammatory diseases of the retina, and proliferative vitreoretinopathy. The corneal neovascularization to be treated or inhibited may be caused by trauma, chemical burns and corneal transplantation. The iris neovascularization to be treated or inhibited may be associated with diabetic retinopathy, vein occlusion, ocular tumor and retinal detachment. The retinal neovascularization to be treated or inhibited may be associated with diabetic retinopathy, vein occlusion, sickle cell retinopathy, retinopathy of prematurity, retinal detachment, ocular ischemia and trauma. The intravitreal neovascularization to be treated or inhibited may be associated with diabetic retinopathy, vein occlusion, sickle cell retinopathy, retinopathy of prematurity, retinal detachment, ocular ischemia and trauma. The choroidal neovascularization to be treated or inhibited may be associated with retinal or subretinal disorders of age-related macular degeneration, presumed ocular histoplasmosis syndrome, myopic degeneration, angioid streaks and ocular trauma.

### INCORPORATION BY REFERENCE

The entire disclosure of each of the publications and patent documents referred to herein is incorporated by reference in its entirely for all purposes to the same extent as if the teachings of each individual publication or patent document were included herein.

### EQUIVALENTS

The invention may be embodied in other specific forms without departing from the spirit of essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

What is claimed is:

1. A transscleral drug delivery device for delivering a drug into a mammalian eye, the device comprising:
(a) a dome member having a base region, the dome member defining a cavity for receiving the drug, and
(b) a base plate attached to the base region, the base plate having a sclera-contacting surface for attaching the device to a scleral surface of the eye, the base plate defining at least one drug outlet port to provide fluid flow communication between the cavity and the scleral surface of the eye when the device is attached to the eye, the drug outlet port comprising at least 25% of the footprint of the base region.

2. The device of claim 1, wherein the base plate has a first diameter and defines at least one drug outlet port having a second diameter, and wherein the second diameter is at least one half of the first diameter.

3. The device of claim 1, wherein the dome member further defines a drug inlet port for introducing the drug into the cavity.

4. The device of claim 3, wherein at least a portion of the dome member is substantially impenetrable to a needle inserted through the drug inlet port.

5. The device of claim 3, wherein at least a portion of the base plate is substantially impenetrable to a needle inserted through the drug inlet port.

6. The device of claim 3 further comprising a puncture guard for preventing a needle inserted through the drug inlet port from contacting the scleral surface of the eye.

7. The device of claim 6, wherein the puncture guard is disposed adjacent to at least one surface of the base plate.

8. The device of claim 6, wherein the puncture guard is disposed adjacent to at least one surface of the dome member.

9. The device of claim 6, wherein the puncture guard is fabricated from a rigid material

10. The device of claim 9, wherein the rigid material comprises a metal.

11. The device of claim 1, wherein the base plate is integral with the dome member.

12. The device of claim 1, wherein at least one of the dome member and the base plate is fabricated from a biocompatible, non-biodegradable material.

13. The device of claim 12, wherein the biocompatible, non-biodegradable material is a metal.

14. The device of claim 1, further comprising a drug disposed within the cavity.

15. A transscleral drug delivery device for delivering a drug into a mammalian eye, the device comprising:
(a) a dome member having a base region, the dome member defining a cavity for receiving the drug and at least one drug inlet port for introducing the drug into the cavity;
(b) a base plate attached to the base region, the base plate having a sclera-contacting surface for attaching the device to a scleral surface of the eye and defining a drug outlet port to provide fluid communication between the cavity and the scleral surface of the eye when the device is attached to the scleral surface; and
(c) a puncture guard for preventing a needle inserted through the drug inlet port from contacting the scleral surface.

16. The device of claim 15, wherein the puncture guard is attached to at least one surface of the base plate.

17. The device of claim 15, wherein the puncture guard is attached to at least one surface of the dome member.

18. The device of claim 15, wherein the puncture guard is fabricated from a rigid material.

19. The device of claim 18, wherein the rigid material comprises a metal.

20. The device of claim 18, wherein the base plate is integral with the dome member.

21. The device of claim 18, wherein at least one of the dome member and the base plate is fabricated from a biocompatible, non-biodegradable material.

22. The device of claim 21, wherein the biocompatible, non-biodegradable material is a metal.

23. The device of claim 15, further comprising a drug disposed within the cavity.

24. A transscleral drug delivery device for delivering a drug into a mammalian eye, the device comprising:
(a) a dome member having a base region, the dome member defining a cavity for receiving the drug and at least one drug inlet port for introducing the drug into the cavity, the drug inlet port configured to prevent a needle inserted therethrough from contacting a scleral surface of the eye when the device is attached to the eye; and
(b) a base plate attached to the base region, the base plate having a sclera-contacting surface for attaching the device to the scleral surface and defining a drug outlet port to provide fluid flow communication between the cavity and the scleral surface when the device is attached to the eye

25. The device of claim 24, wherein the drug inlet port comprises an aperture defined by the dome member and having an axis orthogonal to the aperture, the axis not intersecting the base plate.

26. The device of claim 25, wherein the axis is substantially parallel to the base plate.

27. The device of claim 24, wherein the drug inlet port comprises a generally tubular member defining a lumen having a central longitudinal axis and disposed in an aperture defined by the dome member, the central longitudinal axis of the lumen not intersecting the base plate.

28. The device of claim 27, wherein the central longitudinal axis of the lumen is substantially parallel to the base plate.

29. The device of claim 24, further comprising a drug disposed within the cavity.

30. A method of delivering a drug into a mammalian eye, the method comprising:
(a) attaching the drug delivery device of claim 1 to a scleral surface of the eye; and
(b) permitting drug disposed within the dome member to exit the cavity and contact the scleral surface.

31. The method of claim 30 further comprising the step of prior to or after step (a) introducing drug into the cavity.
